(19) United States
(12) Patent Application Publication
(10) Pub. No.: US 2023/0404036 A1
(43) Pub. Date: Dec. 21, 2023

(54) METHOD, SYSTEM, AND COMPUTER PROGRAM PRODUCT FOR PROVIDING SOLUTION BY MONITORING STATE OF ANIMAL

(71) Applicant: DD CARES CO., LTD., Seongnam-si, Gyeonggi-so (KR)

(72) Inventor: Sang Hyun KIM, Seongnam-si (KR)

(73) Assignee: DD CARES CO., LTD., Seongnam-si, Gyeonggi-do (KR)

(21) Appl. No.: 18/027,715

(22) PCT Filed: Sep. 23, 2020
(86) PCT No.: PCT/KR2020/012820

§ 371 Date: Mar. 22, 2023

(51) Int. Cl. A01K 29/00

(52) U.S. Cl. CPC A01K 29/005; A01K 27/001

(57) ABSTRACT

Provided is a system for monitoring the condition of an animal to provide a solution. The system of the disclosure may include a wearable device worn on a body part of an animal to collect real-time condition information regarding the animal, and a user terminal communicating with the wearable device. The user terminal may receive real-time condition information from the wearable device, determine the type of health abnormality of animal based on the real-time condition information, and provide a solution according to the type of health abnormality.

### TECHNICAL FIELD

The present disclosure relates to a method, a system, and a computer program product for monitoring the condition of animals to provide solutions.

### BACKGROUND ART

The population that recognizes and manages animals, especially dogs and cats, as companion animals is continuously increasing. Recently, a large number of animals suffer from obesity, arthritis, separation anxiety, skin diseases, and allergies, but, due to limitations in communication with animals, it is difficult to detect health problems at the time of initial symptoms, and thus health of animals is deteriorated and treatment costs increase.

There are various ways to manage animals. One of the common method of managing animals is to provide simple management functions like simply providing a video or putting food in a container for an animal left alone while an owner is not at home.

Therefore, beyond common animal management methods, a technology that can help in managing the health of animals and preventing diseases by monitoring the health conditions of animals in real time is demanded.

### DESCRIPTION OF EMBODIMENTS

### Technical Problem

The present disclosure provides a method, a system, and a computer program product for monitoring the condition of animals and provide solutions. The technical goals to be achieved by the present embodiment are not limited to the technical goals as described above, and other technical problems may be inferred from the following embodiments.

### Solution to Problem

According to an embodiment of the present disclosure, a system for monitoring a condition of an animal to provide a solution, the system includes a wearable device worn on a body part of an animal to collect real-time condition information regarding the animal, and a user terminal communicating with the wearable device, wherein the user terminal receives the real-time condition information from the wearable device, determines a type of health abnormality of the animal based on the real-time condition information, and provides a solution according to the type of health abnormality.

In an embodiment, at least one task helpful to health of the animal may be provided as a solution, based on the type of health abnormality.

In an embodiment, the user terminal may provide a reward to a user according to performance of the at least one task.

In an embodiment, the user terminal may receive new real-time condition information from the wearable device after the at least one task is performed, and update the at least one task based on performance of the at least one task and the new real-time condition information.

In an embodiment, the user terminal may provide feed information helpful to health of the animal as a solution, based on the type of health abnormality.

In an embodiment, the user terminal may receive an input from a user to obtain basic information regarding the animal, and determine the type of health abnormality of the animal based on the real-time condition information and the basic information.

In an embodiment, the user terminal may provide at least one task helpful to health of the animal as a solution, based on the type of health abnormality and the basic information.

In an embodiment, the user terminal may verify the basic information by comparing the real-time condition information with the basic information.

According to another embodiment of the present disclosure, there is provided a computer program product for monitoring a condition of an animal to provide a solution, the computer program product including one or more computer-readable recording media having stored thereon a program for performing receiving real-time condition information regarding the animal from a wearable device worn on a body part of the animal, determining a type of health abnormality of the animal based on the real-time condition information, and providing a solution according to the type of health abnormality.

According to another embodiment of the present disclosure, a method of monitoring a condition of an animal to provide a solution, the method includes receiving real-time condition information regarding the animal from a wearable device worn on a body part of the animal, determining a type of health abnormality of the animal based on the real-time condition information, and providing a solution according to the type of health abnormality.

### Advantageous Effects of Disclosure

According to an above-stated solution of the present disclosure, it is possible to more efficiently manage the health of an animal by receiving real-time condition information regarding an animal from a wearable device and providing a type of health abnormality of the animal and a solution thereof based on the real-time condition information.

According to another solution of the present disclosure, a solution that best matches the current health condition of an animal may be provided by updating contents of a solution based on performance of a task provided as the solution and new real-time condition information received after the task is performed.

According to another solution of the present disclosure, it is possible to more accurately determine the type of health abnormality of an animal by considering basic information regarding the animal as well as real-time condition information regarding the animal.

### BRIEF DESCRIPTION OF DRAWINGS

FIG. 1 is a diagram showing a system including a user terminal and a wearable device according to an embodiment.

FIG. 2 is a diagram showing an example of transmitting real-time condition information regarding an animal from a wearable device to a user terminal according to an embodiment.

FIG. 3 is a diagram for describing an example of determining the type of health abnormality of an animal based on real-time condition information regarding the animal, according to an embodiment.

FIG. 4 is a diagram for describing an example of providing a task as a solution according to an embodiment.

FIG. 5 is a diagram for describing an example of providing a feed information as a solution according to an embodiment.

FIGS. 6A to 6B are diagrams for describing an example of updating a task in consideration of performance of the task, according to an embodiment.

FIGS. 7A to 7B are diagrams for describing an example of providing a task in consideration of basic information regarding an animal, according to an embodiment.

FIG. 8 is a flowchart of a method of providing a solution by monitoring the condition of an animal, according to an embodiment.

FIG. 9 is a block diagram of an external server according to an embodiment.

### BEST MODE

Provided is a system for monitoring the condition of an animal to provide a solution. The system of the disclosure may include a wearable device worn on a body part of an animal to collect real-time condition information regarding the animal, and a user terminal communicating with the wearable device. The user terminal may receive real-time condition information from the wearable device, determine the type of health abnormality of animal based on the real-time condition information, and provide a solution according to the type of health abnormality.

### Mode of Disclosure

Hereinafter, exemplary embodiments of the disclosure will be described in detail with reference to the accompanying drawings. The disclosure may, however, be embodied in many different forms and should not be construed as limited to the embodiments set forth herein. In order to clearly illustrate the disclosure, parts not related to the description are omitted, and like parts are denoted by like reference numerals throughout the specification.

Throughout the specification, it will be understood that when a portion is referred to as being “connected to” another portion, it can be “directly connected to” the other portion or “electrically connected to” the other portion via another element. In addition, unless explicitly described to the contrary, the word “comprise” and variations such as “comprises” or “comprising” will be understood to imply the inclusion of stated elements but not the exclusion of any other elements.

Hereinafter, the disclosure will be described in detail with reference to accompanying drawings.

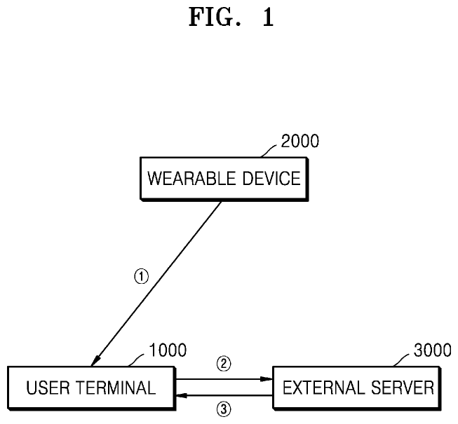

FIG. 1 is a diagram showing a system including a user terminal and a wearable device according to an embodiment.

A system according to an embodiment may include a user terminal 1000 and a wearable device 2000. In an embodiment, the system may further include an external server 3000.

The user terminal 1000, the wearable device 2000, and the external server 3000 may perform communication with one another by using a network. For example, the network is a comprehensive data communication network, which includes a local area network (LAN), a wide area network (WAN), a value added network (VAN), a mobile radio communication network, a satellite communication network, and mutual combinations thereof, enables smooth communication between the network constituent entities shown in FIG. 1, and may include a wired internet, a wireless internet, and a mobile wireless communication network. In an embodiment, wireless communication may include, but is not limited to, for example, wireless LAN (Wi-Fi), Bluetooth, Bluetooth low energy, Zigbee, Wi-Fi Direct (WFD), ultra wideband (UWB), infrared data association (IrDA), and Near Field Communication (NFC).

The user terminal 1000 may include, but is not limited to, any one of a smartphone, a tablet PC, a PC, a smart TV, a mobile phone, a personal digital assistant (PDA), a laptop computer, a media player, a micro server, a global positioning system (GPS) device, an e-book reader, a digital broadcasting terminal, a navigation device, a kiosk, an MP3 player, a digital camera, a home appliance, a device having a camera, and other mobile or non-mobile computing devices.

The wearable device 2000 may be worn on a body part of an animal to collect real-time condition information of the animal. The wearable device 2000 may include a plurality of sensors. For example, the wearable device 2000 may include at least one of an electromyography (EMG) sensor, an electrodermal activity sensor, a skin temperature measuring device, a blood volume pulse measuring device, an electrocardiogram (ECG) sensor, a respiration sensor, a blood pressure measuring device, and a heart rate measuring device. In an embodiment, the wearable device 2000 may include a 3-axis accelerometer, a 6-axis gyroscope & accelerometer, and a 9-axis gyroscope & accelerometer & geomagnetic sensor.

The external server 3000 may communicate with the user terminal 1000 and the wearable device 2000 through a network.

According to an embodiment, referring to a first arrow, the wearable device 2000 may transmit real-time condition information regarding an animal to the user terminal 1000. The wearable device 2000 may transmit real-time condition information regarding an animal to the user terminal 1000 through a communication method like Wi-Fi, Bluetooth, and Bluetooth low energy. Referring to a second arrow, the user terminal 1000 may transmit real-time condition information to the external server 3000 and request analysis thereof. The external server 3000 may analyze the real-time condition information to determine the type of an health abnormality of the animal and determine a solution according to the type of the health abnormality. Referring to a third arrow, the external server 3000 may transmit the type of the health abnormality of the animal and a solution thereof to the user terminal 1000, and the user terminal 1000 may provide the type of the health abnormality of the animal and the solution thereof to a user. The user terminal 1000 and the external server 3000 may exchange data through wired Internet, wireless Internet, or a mobile wireless communication network.

According to another embodiment, referring to the first arrow, real-time condition information regarding an animal obtained by the wearable device 2000 may be transmitted to the user terminal 1000. At this time, instead of transmitting the real-time condition information to the external server 3000, the user terminal 1000 may analyze the real-time condition information on its own to determine the type of an health abnormality of the animal and provide a soliton according to the type of the health abnormality to a user.

According to another embodiment, although not shown in FIG. 1, the wearable device 2000 may transmit real-time condition information directly to the external server 3000 and request analysis of the real-time condition information, without going through the user terminal 1000. In this case, the external server 3000 may transmit the type of the health abnormality of the animal and a solution thereof to the user terminal 1000, and the user terminal 1000 may provide the type of the health abnormality of the animal and the solution thereof to a user. The user terminal 1000, the wearable device 2000, and the external server 3000 may exchange data through wired Internet, wireless Internet, or a mobile wireless communication network.

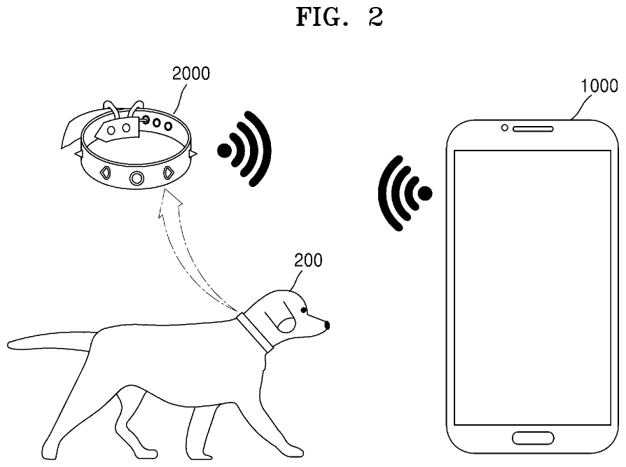

FIG. 2 is a diagram showing an example of transmitting real-time condition information regarding an animal from a wearable device to a user terminal according to an embodiment.

Referring to FIG. 2, a wearable device 2000 may be worn on a body part of an animal 200 to collect real-time condition information regarding the animal 200.

In one embodiment, the real-time condition information may be information indicating whether the animal 200 is currently in an active state or a sleeping state. More specifically, the real-time condition information may be information indicating whether the animal 200 is in a play state, an active state, a resting state, or a sleeping state. However, the type of real-time condition information is not limited thereto.

The wearable device 2000 may include at least one sensor, and the wearable device 2000 may collect real-time condition information regarding the animal 200 based on a sensed value of the at least one sensor.

For example, the wearable device 2000 may include at least one of an EMG sensor, an electrodermal activity sensor, a skin temperature measuring device, a blood volume pulse measuring sensor, an ECG sensor, a respiration sensor, a blood pressure measuring device, a heart rate measuring device, a 3-axis accelerometer, a 6-axis gyroscope/accelerometer, and a 9-axis gyroscope/accelerometer/geomagnetic sensor. The EMG sensor refers to a sensor that detects an action potential of a muscle. The electrodermal activity sensor refers to a sensor that measures skin conductivity. The skin temperature measuring device may include a sensor that detects the temperature of the skin surface. The blood volume pulse measuring sensor refers to a device that measures the amount of blood flowing in a blood vessel. The ECG sensor refers to a sensor that senses an electrical potential related to heartbeat on a body surface. The respiration sensor refers to a sensor that measures how much and quickly one breathes. The heart rate measuring device refers to a device that measures the number of times the heart beats per unit time. The 3-axis accelerometer measures acceleration, the 6-axis gyroscope/accelerometer measures acceleration and angular velocity, and the 9-axis gyroscope/accelerometer/geomagnetic sensor measures acceleration and angular velocity and detects geomagnetism.

The user terminal 1000 may receive real-time condition information regarding the animal 200 from the wearable device 2000.

The user terminal 1000 may determine the type of health abnormality of the animal 200 based on real-time condition information. For example, the type of health abnormality of the animal 200 may include arthritis, skin disease, allergy, separation anxiety, etc., but is not limited thereto.

In an embodiment, the user terminal 1000 may provide a solution according to the type of health abnormality of the animal 200. The solution may be at least one task helpful for the health of the animal 200. Alternatively, the solution may be feed information helpful for the health of the animal 200.

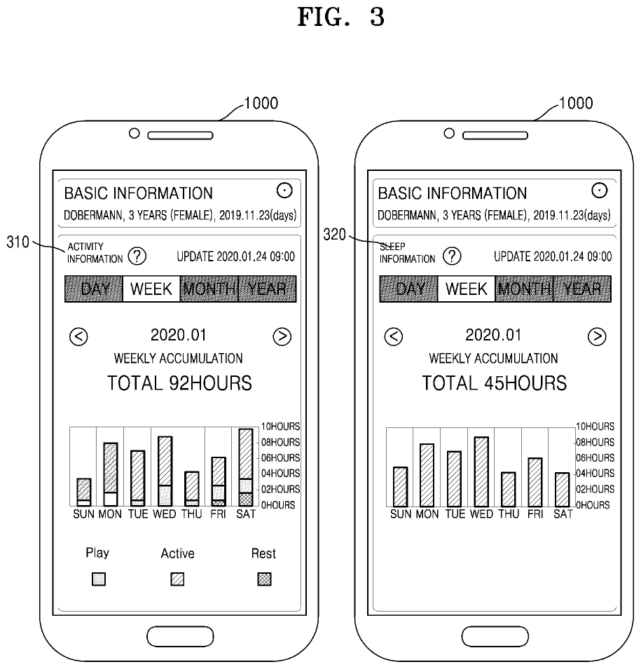

FIG. 3 is a diagram for describing an example of determining the type of health abnormality of an animal based on real-time condition information regarding the animal, according to an embodiment.

The user terminal 1000 may receive real-time condition information regarding an animal from a wearable device and determine the type of health abnormality of the animal based on real-time condition information regarding the animal.

Referring to FIG. 3, the user terminal 1000 may receive real-time condition information regarding an animal from a wearable device, accumulate received real-time condition information on a daily, weekly, monthly, and yearly basis, and provide accumulated information to a user.

According to an embodiment, the user terminal 1000 may aggregate accumulated time for each of a play state, an active state, and a resting state in the real-time condition information and provide aggregated times to a user. In an embodiment, the user terminal 1000 may accumulate time for a sleeping state in the real-time condition information and provide accumulated time to a user.

Meanwhile, the user terminal 1000 may not only provide real-time condition information to a user, but also determine the type of health abnormality of an animal based on the real-time condition information.

For example, when a result of analyzing activity information 310 in the real-time condition information indicates that the time corresponding to a play state of an animal is equal to or greater than a critical time, the user terminal 1000 may determine the type of health abnormality of the animal as separation anxiety.

Alternatively, when a result of analyzing the activity information 310 indicates that the time corresponding to a resting state of an animal is equal to or greater than a critical time, the user terminal 1000 may determine the type of health abnormality of the animal as arthritis.

However, the user terminal 1000 may determine the type of health abnormality of an animal in consideration of various sensed values received from the wearable device as well as times corresponding to the play state, the active state, the resting state, and the sleeping state of the animal. For example, the user terminal 1000 may determine the type of health abnormality of an animal in consideration of the sensed values received from an EMG sensor, an electrodermal activity sensor, a skin temperature measuring device, a blood pressure measuring device, an accelerometer, etc. of the wearable device.

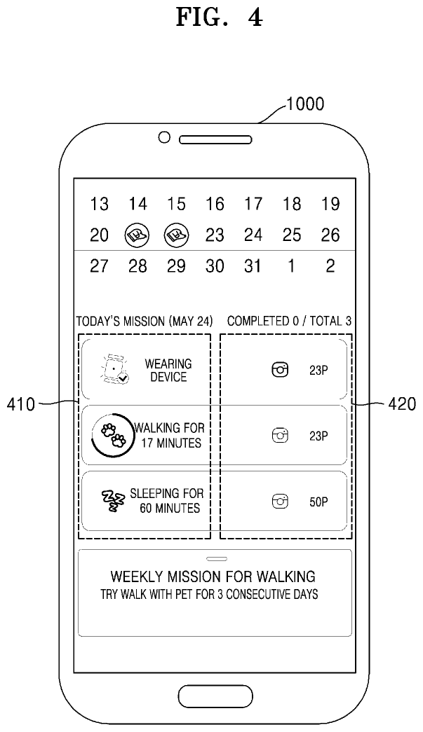

FIG. 4 is a diagram for describing an example of providing a task as a solution according to an embodiment.

The user terminal 1000 may provide at least one task 410 helpful for the health of an animal as a solution based on the type of health abnormality of the animal. The task 410 provided by the user terminal 1000 may include the number of times of walking, walking time, sleeping time, etc.

Referring to FIG. 4, the user terminal 1000 may provide tasks like device wearing, walking time, and sleeping time. In detail, when the type of health abnormality of an animal is obesity, the user terminal 1000 may provide a minimum walking time as the task 410. Alternatively, when the type of health abnormality of an animal is arthritis, the user terminal 1000 may provide a resting time as the task 410.

In an embodiment, the user terminal 1000 may provide a reward 430 to a user according to performance of at least one task. For example, from among today's tasks, 23 points (P) may be provided as a reward when a task of ‘wearing device’ is performed, 23P may be provided as a reward when a task of ‘walking for 17 minutes’ is performed, and 50p may be provided as a reward when a task of ‘sleeping for 60 minutes’ is performed.

According to an embodiment, rewards may be provided differentially. Rewards may be provided differentially according to task performance rates. When 50P is provided as a reward for completing the task of ‘sleeping for 60 minutes’, 25P may be provided as a reward for completing only ‘sleeping for 30 minutes’. In an embodiment, rewards may be provided differentially according to performance rates of tasks. Depending on the content of tasks, certain tasks may exhibit high performance rates, while other tasks may exhibit low performance rates. Since all tasks are helpful for animal health, to increase the performance rates for tasks with low performance rates, more rewards may be provided when the tasks with low performance rates are performed.

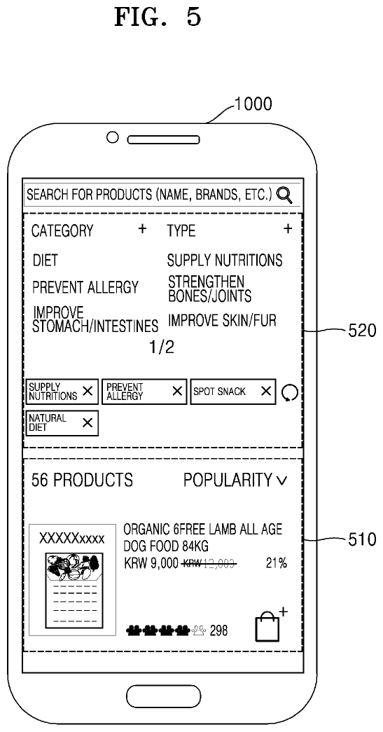

Meanwhile, a user may use rewards in various ways. According to an embodiment, a user may redeem reward points for cash. According to another embodiment, a user may use rewards points to purchase a feed product offered by a solution. According to another embodiment, a user may use rewards points to purchase an insurance plan offered by a solution. Cases where a feed product and an insurance plan are provided as solutions will be described below with reference to FIG. 5.

FIG. 5 is a diagram for describing an example of providing a feed information as a solution according to an embodiment.

The user terminal 1000 may provide feed information 510 helpful for the health of an animal as a solution based on the type of health abnormality of an animal. The feed information 510 provided by the user terminal 1000 may include necessary nutrient information and feed product information.

In detail, when the type of health abnormality of an animal is obesity, the user terminal 1000 may provide nutrition information for diet or information regarding a feed product for diet as the feed information 510. Alternatively, when the type of health abnormality of an animal is arthritis, the user terminal 1000 may provide nutrient information for strengthening bones/joints or a feed product for improving arthritis as the feed information 510.

According to an embodiment, when a user purchases a feed product provided as the feed information 510, the user terminal 1000 may provide a reward.

According to an embodiment, a user may purchase a feed product provided as the feed information 510 by using a reward. Referring to FIG. 4, the user terminal 1000 may provide a reward to a user according to performance of at least one task. A user may purchase a feed product provided as the feed information 510 by using a reward obtained by performing a task.

Meanwhile, a user may select an additional condition 520. The user terminal 1000 may receive an input for selecting the additional condition 520 by a user. When the additional condition 520 is determined, the user terminal 1000 may provide only feed products that satisfy the additional condition 520 from among the food products provided as the feed information 510 to the user.

For example, when the type of health abnormality of an animal is arthritis, the user terminal 1000 may provide feed products for improving arthritis by strengthening bones/joints as the feed information 510. At this time, when ‘natural diet’ is determined as the additional condition 520, the user terminal 100 may provide the user only with feed products satisfying the condition ‘natural diet’ from among the feed products for improving arthritis.

Although not shown in FIG. 5, the user terminal 1000 may provide animal insurance information as a solution based on the type of health abnormality of an animal. Animal insurance information provided by the user terminal 1000 may include insurance plan information, etc. For example, when the type of health abnormality of an animal is arthritis, the user terminal 1000 may provide a user with an insurance plan including arthritis coverage. A user may use rewards points to purchase an insurance plan offered by a solution. In an embodiment, when a user purchases an insurance plan offered as a solution, the user terminal 1000 may provide a reward to the user.

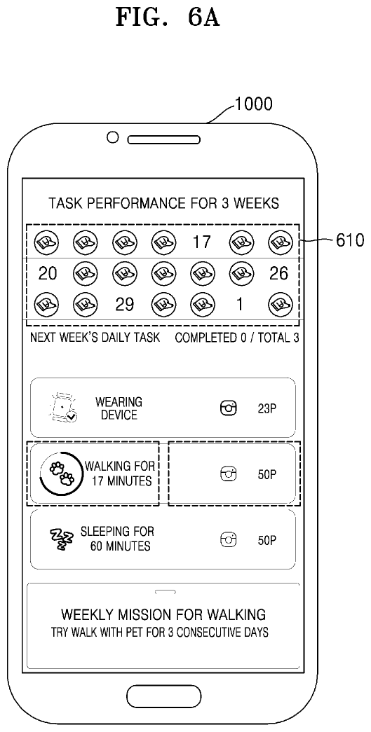

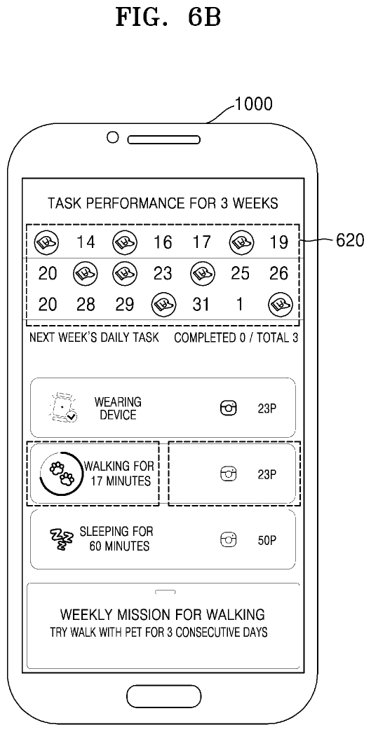

FIGS. 6A to 6B are diagrams for describing an example of updating a task in consideration of performance of the task, according to an embodiment.

After at least one task is performed, the user terminal 1000 may receive new real-time condition information from a wearable device and update the at least one task based on a performance result for the at least one task and the new real-time condition information.

For example, when the type of health abnormality of an animal is obesity, ‘walking for 20 minutes’ may be provided as a task for 3 weeks.

Referring to FIG. 6A, it can be seen that a 3-week task performance rate 610 is very high. In an embodiment, as a result of analyzing new real-time condition information received from the wearable device after performing a task, the user terminal 1000 may derive a result that the animal moves more actively than before performing the task.

The user terminal 1000 may provide a task for ‘walking’ for a time reduced from 20 minutes to 17 minutes after determining that the degree of obesity of the animal is improved based on the task performance and the new real-time condition information. In an embodiment, the user terminal 1000 may provide more rewards 50P to a user when the task performance rate is high.

On the other hand, referring to FIG. 6B, it may be seen that a 3-week task performance rate 620 is very low. In an embodiment, as a result of analyzing new real-time condition information received from the wearable device after performing a task, the user terminal 1000 may derive a result that the animal moves less actively than before performing the task.

The user terminal 1000 may provide a task for ‘walking’ for a time increased from 20 minutes to 26 minutes after determining that the degree of obesity of the animal not is improved based on the task performance and the new real-time condition information.

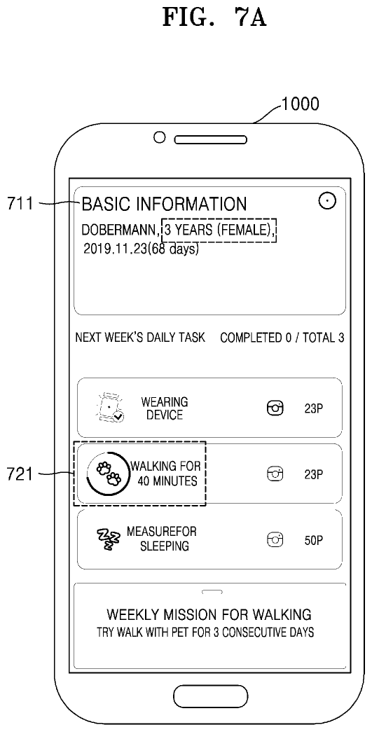

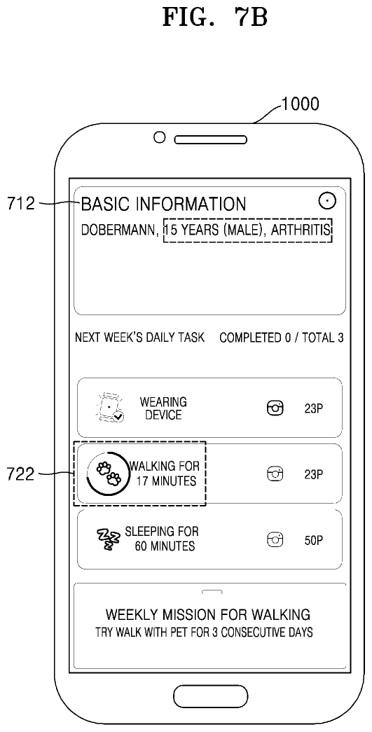

FIGS. 7A to 7B are diagrams for describing an example of providing a task in consideration of basic information regarding an animal, according to an embodiment.

The user terminal 1000 may obtain basic information regarding an animal by receiving an input from a user. Basic information regarding an animal may include information regarding the breed, the age, the sex, the neutered status, the disease, the allergy, etc. of the animal, but information that may be included in the basic information is not limited thereto.

As described above with reference to FIG. 3, the user terminal 1000 may receive real-time condition information regarding an animal from a wearable device and determine the type of health abnormality of the animal based on the real-time condition information regarding the animal.

According to an embodiment, the user terminal 1000 may determine the type of health abnormality of an animal in consideration of basic information regarding the animal as well as real-time condition information regarding the animal.

For example, when a result of analyzing activity information in real-time condition information indicates that the time corresponding to a resting state of an animal is equal to or greater than a critical time, the user terminal 1000 generally may determine the type of health abnormality of the animal as arthritis. On the other hand, when information indicating that the animal is 2 years old is input as basic information regarding the animal, since it is unlikely that a 2-year old animal is suffering from arthritis, the user terminal 1000 may determine the type of health abnormality of the animal as fatigue.

According to the disclosure, by determining the type of health abnormality in consideration of real-time condition information regarding an animal as well as basic information regarding the animal, it is possible to determine the type of health abnormality of the animal more accurately.

As described above with reference to FIG. 4, the user terminal 1000 may provide at least one task helpful for health of an animal as a solution based on the type of health abnormality of the animal. In an embodiment, as described above with reference to FIG. 5, the user terminal 1000 may provide feed information and animal insurance information helpful for health of an animal as a solution based on the type of health abnormality of the animal.

According to an embodiment, the user terminal 1000 may provide at least one task as a solution in consideration of the type of health abnormality of an animal as well as basic information regarding the animal. Hereinafter, descriptions will be given on the assumption that the type of health abnormal of an animal is obesity.

Referring to FIG. 7A, information indicating that an animal is 3 years old may be input as basic information 711 regarding the animal. Since the type of health abnormality of the animal is obesity, ‘walking for 40 minutes’ mat be provided as today's task.

On the other hand, referring to FIG. 7B, information indicating that an animal is 15 years old and has arthritis may be input as basic information 712 regarding the animal. Since the type of health abnormality of the animal is obesity, ‘walking for 40 minutes’ is generally provided as today's task. However, considering that the basic information 712 regarding the animal, a long walk may aggravate the arthritis. The user terminal 1000 may provide ‘walking for 17 minutes’, which is shorter than the general case, as today's task by considering the type of health abnormality of the animal as well as the basic information 712 regarding the animal.

According to another embodiment, the user terminal 1000 may provide feed information as a solution in consideration of the type of health abnormality of an animal as well as basic information regarding the animal. For example, information indicating that an animal is allergic to a specific ingredient may be input as basic information regarding the animal. In this case, the user terminal 1000 may provide a user with only feed products that do not contain the specific ingredient from among feed products for diet.

According to another embodiment, the user terminal 1000 may provide animal insurance information as a solution in consideration of the type of health abnormality of an animal as well as basic information regarding the animal. For example, as basic information regarding an animal, information indicating that the animal is 15 years old and information about the dog breed of the animal may be input. In this case, the user terminal 1000 may provide a user with insurance plans that cover general diseases when the specific dog breed gets old.

According to the disclosure, a more accurately customized solution for each animal mat be provided by providing a solution in consideration of the type of health abnormality of the animal as well as basic information regarding the animal.

Meanwhile, the user terminal 1000 may compare real-time condition information with basic information to verify whether the basic information is correct.

According to an embodiment, the user terminal 1000 may obtain information indicating that an animal has a disease A as basic information regarding the animal. As a result of analyzing real-time condition information regarding the animal, the user terminal 1000 may determine that the type of health abnormality of the animal is more likely to correspond to a disease B than the disease A. As such, there is a possibility that basic information regarding the animal known by a user is wrong or that the basic information has changed over time.

Real-time condition information according to the disclosure is information that most accurately shows the current condition of an animal. According to the disclosure, by comparing real-time condition information and basic information, a user may be provided with more accurate basic information regarding an animal.

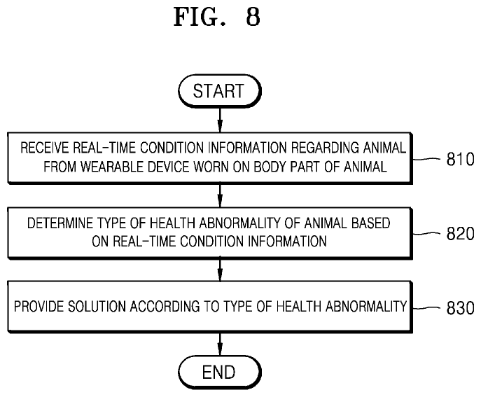

FIG. 8 is a flowchart of a method of providing a solution by monitoring the condition of an animal, according to an embodiment.

Referring to FIG. 8, in operation 810, a processor may receive real-time condition information regarding an animal from a wearable device worn on a body part of the animal.

The wearable device may include at least one sensor, and the wearable device may collect real-time condition information regarding the animal based on a sensed value of the at least one sensor.

In one embodiment, the real-time condition information may be information indicating whether the animal is currently in an active state or a sleeping state. More specifically, the real-time condition information may be information indicating whether the animal is in a play state, an active state, a resting state, or a sleeping state. However, the type of real-time condition information is not limited thereto.

In operation 820, the processor may determine the type of health abnormality of the animal based on the real-time condition information.

For example, the type of health abnormality may include arthritis, skin disease, allergy, separation anxiety, etc., but is not limited thereto.

For example, when a result of analyzing activity information in real-time condition information indicates that the time corresponding to a play state of an animal is equal to or greater than a critical time, the processor may determine the type of health abnormality of the animal as separation anxiety. Alternatively, when a result of analyzing the activity information indicates that the time corresponding to a resting state of an animal is equal to or greater than a critical time, the processor may determine the type of health abnormality of the animal as arthritis.

According to an embodiment, the processor may obtain basic information regarding an animal by receiving an input from a user and determine the type of health abnormality of the animal based on real-time condition information and the basic information.

Basic information regarding an animal may include information regarding the breed, the age, the sex, the neutered status, the disease, the allergy, etc. of the animal, but information that may be included in the basic information is not limited thereto.

For example, when a result of analyzing activity information in real-time condition information indicates that the time corresponding to a resting state of an animal is equal to or greater than a critical time, the processor generally may determine the type of health abnormality of the animal as arthritis. On the other hand, when information indicating that the animal is 2 years old is input as basic information regarding the animal, since it is unlikely that a 2-year old animal is suffering from arthritis, the processor may determine the type of health abnormality of the animal as fatigue.

According to an embodiment, the processor may verify basic information by comparing real-time condition information with the basic information. The processor may obtain information indicating that an animal has a disease A as basic information regarding the animal. As a result of analyzing real-time condition information regarding the animal, the processor may determine that the type of health abnormality of the animal is more likely to correspond to a disease B than the disease A.

In operation 830, the processor may provide a solution according to the type of health abnormality.

According to an embodiment, the processor may provide at least one task helpful for the health of an animal as a solution based on the type of health abnormality of the animal.

In an embodiment, the processor may provide a reward to a user according to performance of the at least one task.

In an embodiment, after the at least one task is performed, the processor may receive new real-time condition information from a wearable device and update the at least one task based on a performance result for the at least one task and the new real-time condition information.

According to another embodiment, the processor may provide feed information helpful for the health of an animal as a solution based on the type of health abnormality of an animal.

Meanwhile, when basic information regarding an animal is obtained from a user, the processor may provide at least one task or feed information useful for the health of the animal as a solution based on the type of health abnormality and the basic information.

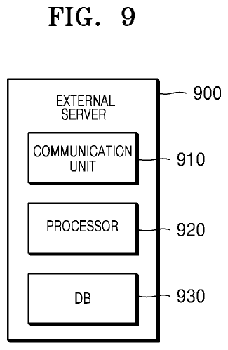

FIG. 9 is a block diagram of an external server according to an embodiment.

Referring to FIG. 9, an external server 900 may include a communication unit 910, a processor 920, and a DB 930. Only components related to the embodiment are shown in the external server 900 of FIG. 9. Therefore, one of ordinary skill in the art will understand that other general-purpose components may be further included in addition to the components shown in FIG. 9.

The communication unit 910 may include one or more components that enable wired/wireless communication between user terminals and payment information providing servers. For example, the communication unit 910 may include at least one of a short-range communication unit (not shown), a mobile communication unit (not shown), and a broadcast reception unit (not shown).

The DB 930 is hardware for storing various data processed in the external server 900 and may store programs for processing and controlling the processor 920.

The DB 930 may include a random access memory (RAM), such as a dynamic random access memory (DRAM) and a static random access memory (SRAM), a read-only memory (ROM), an electrically erasable programmable read-only memory (EEPROM), a CD-ROM, a Blu-ray or another optical disc storage, a hard disk drive (HDD), a solid state drive (SSD), or a flash memory.

The processor 920 controls the overall operation of the external server 900. For example, the processor 920 may generally control an input unit (not shown), a display (not shown), the communication unit 910, and the DB 930 by executing programs stored in the DB 930. The processor 920 may control the operation of the external server 900 by executing programs stored in the DB 930.

The processor 920 may be implemented by using at least one of application specific integrated circuits (ASICs), digital signal processors (DSPs), digital signal processing devices (DSPDs), programmable logic devices (PLDs), field programmable gate arrays (FPGAs), controllers, micro-controllers, microprocessors, and other electrical units for performing functions.

The external server 900 may communicate with a user terminal and a wearable device worn on a body part of an animal through the communication unit 910.

The communication unit 910 may receive real-time condition information regarding an animal from a user terminal or a wearable device.

Real-time condition information regarding an animal may be stored in the DB 930. The processor 920 may determine the type of health abnormality of an animal based on real-time condition information regarding the animal. In an embodiment, the processor 920 may determine a solution according to the type of health abnormality.

The DB 930 may store the type of health abnormality type according to real-time condition information regarding an animal and a solution according to the type of health abnormality, which are determined by the processor 920.

The communication unit 910 may transmit the type of health abnormality and a solution to a user terminal or a wearable device.

Various embodiments of the present disclosure are limited to a case where a wearable device is worn on a body part of an animal, but the embodiments may be equally applicable to a case where a wearable device is worn on a body part of a person. In detail, a wearable device may be worn on a body part of a person to collect real-time condition information regarding the person, and a user terminal may receive the real-time condition information regarding the person, determine the type of health abnormality of the person, and provide a solution according to the type of health abnormality.

Various embodiments of the present disclosure may be implemented as software (e.g., a program) including one or more instructions stored in a machine-readable storage medium. For example, a processor of a device may invoke at least one instruction from among one or more instructions stored in a storage medium and execute it. This enables the device to be operated to perform at least one function according to the at least one instruction invoked. The one or more instructions may include codes generated by a compiler or codes executable by an interpreter. The machine-readable storage medium may be provided in the form of a non-transitory storage medium. Here, the term ‘non-temporary’ merely means that a storage medium is a tangible device and does not contain signals (e.g., electromagnetic waves), and this term does not discriminate the case where data is stored semi-permanently in a storage medium and the case where data is temporarily stored in a storage medium.

According to an embodiment, the method according to various embodiments of the present disclosure may be included and provided in computer program products. Computer program products may be traded between sellers and buyers as commodities. Computer program products may be distributed in the form of a device-readable storage medium (e.g., compact disc read-only memory (CD-ROM)), on-line (e.g., download or upload) through an application store (e.g., PlayStore™), or directly between two user devices. In the case of online distribution, at least a part of a computer program product may be temporarily stored or temporarily created in a machine-readable storage medium like a memory of a manufacturer's server, an application store server, or a relay server.

In this specification, a “unit” may be a hardware component such as a processor or a circuit, and/or software component executed on the hardware like a processor.

The scope of the disclosure is indicated by the claims which will be described in the following rather than the detailed description of the exemplary embodiments, and it should be understood that the claims and all modifications or modified forms drawn from the concept of the claims are included in the scope of the disclosure.

1. A system for monitoring a condition of an animal to provide a solution, the system comprising:
a wearable device worn on a body part of an animal to collect real-time condition information regarding the animal; and
a user terminal communicating with the wearable device,
wherein the user terminal
receives the real-time condition information from the wearable device,
determines a type of health abnormality of the animal based on the real-time condition information, and
provides a solution according to the type of health abnormality.

2. The system of claim 1, wherein the user terminal provides at least one task helpful to health of the animal as a solution, based on the type of health abnormality.

3. The system of claim 2, wherein the user terminal provides a reward to a user according to performance of the at least one task.

4. The system of claim 2, wherein the user terminal receives new real-time condition information from the wearable device after the at least one task is performed, and updates the at least one task based on performance of the at least one task and the new real-time condition information.

5. The system of claim 1, wherein the user terminal provides feed information helpful to health of the animal as a solution, based on the type of health abnormality.

6. The system of claim 1, wherein the user terminal receives an input from a user to obtain basic information regarding the animal, and determines the type of health abnormality of the animal based on the real-time condition information and the basic information.

7. The system of claim 6, wherein the user terminal provides at least one task helpful to health of the animal as a solution, based on the type of health abnormality and the basic information.

8. The system of claim 6, wherein the user terminal verifies the basic information by comparing the real-time condition information with the basic information.

9. A computer program product for monitoring a condition of an animal to provide a solution, the computer program product comprising one or more computer-readable recording media having stored thereon a program for performing:
receiving real-time condition information regarding the animal from a wearable device worn on a body part of the animal;
determining a type of health abnormality of the animal based on the real-time condition information; and
providing a solution according to the type of health abnormality.

10. A method of monitoring a condition of an animal to provide a solution, the method comprising:
receiving real-time condition information regarding the animal from a wearable device worn on a body part of the animal;
determining a type of health abnormality of the animal based on the real-time condition information; and
providing a solution according to the type of health abnormality.
